(19) United States
(12) Patent Application Publication
(10) Pub. No.: US 2020/0163554 A1
(43) Pub. Date: May 28, 2020

(54) IMAGE GENERATING APPARATUS, IMAGE GENERATING METHOD, AND NON-TRANSITORY COMPUTER-READABLE MEDIUM

(71) Applicant: CANON KABUSHIKI KAISHA, Tokyo (JP)

(72) Inventor: Yoshitaka Baba, Tokyo (JP)

(21) Appl. No.: 16/735,496

(22) Filed: Jan. 6, 2020

Related U.S. Application Data
(63) Continuation of application No. PCT/JP2018/025676, filed on Jul. 6, 2018.

(30) Foreign Application Priority Data
Jul. 13, 2017 (JP) 2017-137181

(51) Int. Cl. A61B 5/00

(52) U.S. Cl. CPC A61B 5/0095

(57) ABSTRACT

An image generating apparatus according to the present invention includes an image data generator configured to generate a plurality of pieces of image data corresponding to a plurality of radiated light beams based on a plurality of the reception signals, a characteristic-information acquiring unit configured to acquire characteristic information indicating characteristics of an image value group of the plurality of pieces of image data at a certain position, and an information acquiring unit configured to acquire information indicating a possibility that a target exists at the certain position based on the characteristic information.

### CROSS-REFERENCE TO RELATED APPLICATIONS

This application is a Continuation of International Patent Application No. PCT/JP2018/025676, filed Jul. 6, 2018, which claims the benefit of Japanese Patent Application No. 2017-137181, filed Jul. 13, 2017, both of which are hereby incorporated by reference herein in their entirety.

### TECHNICAL FIELD

The present invention relates to image generating apparatuses that generate image data derived from a photoacoustic wave generated as a result of light irradiation.

### BACKGROUND ART

A known photoacoustic apparatus generates image data based on a reception signal obtained as a result of receiving an acoustic wave. A photoacoustic apparatus irradiates a subject with pulsed light generated from a light source, and receives an acoustic wave (typically, an ultrasonic wave and also referred to as a photoacoustic wave) generated from subject tissue having absorbed the energy of the pulsed light propagated and diffused through the subject. The photoacoustic apparatus then converts subject information into an image based on the reception signal.

NPL 1 discloses universal back-projection (UBP), which is one of back-projection methods, as a method for converting an initial sound-pressure distribution into an image from a reception signal of a photoacoustic wave.

### CITATION LIST

### Non Patent Literature

NPL 1 “Universal back-projection algorithm for photoacoustic computed tomography”, Minghua Xu and Lihong V. Wang, PHYSICAL REVIEW E 71, 016706 (2005)

If image data is to be generated by back-projecting a reception signal of an acoustic wave, the reception signal is back-projected onto a position other than the position where the acoustic wave is generated and appears as an artifact in the image. Accordingly, it may sometimes be difficult to differentiate whether or not an image within the image is an image of a target (observation target).

An object of the present invention is to provide an image generating apparatus that can facilitate the differentiation of whether the possibility of the existence of a target (observation target) at a certain position within an image is high or low.

### SUMMARY OF INVENTION

An image generating apparatus according to the present invention generates image data based on a reception signal obtained by receiving a photoacoustic wave generated from a subject as a result of radiating light onto the subject, and includes: an image data generator configured to generate a plurality of pieces of image data corresponding to a plurality of radiated light beams based on a plurality of the reception signals obtained by radiating light a plurality of times onto the subject; a characteristic-information acquiring unit configured to acquire characteristic information indicating characteristics of an image value group of the plurality of pieces of image data at a certain position; and an information acquiring unit configured to acquire information indicating a possibility that a target exists at the certain position based on the characteristic information.

Further features of the present invention will become apparent from the following description of exemplary embodiments with reference to the attached drawings.

### BRIEF DESCRIPTION OF DRAWINGS

FIG. 1A is a diagram for explaining a time differentiation process and a positive-negative inversion process according to universal back-projection (UBP).

FIG. 1B is a diagram for explaining the time differentiation process and the positive-negative inversion process according to UBP.

FIG. 1C is a diagram for explaining the time differentiation process and the positive-negative inversion process according to UBP.

FIG. 2A is a diagram for explaining a back projection process according to UBP.

FIG. 2B is a diagram for explaining the back projection process according to UBP.

FIG. 2C is a diagram for explaining the back projection process according to UBP.

FIG. 2D is a diagram for explaining the back projection process according to UBP.

FIG. 2E is a diagram for explaining the back projection process according to UBP.

FIG. 3A illustrates variations in an image value obtained in accordance with UBP.

FIG. 3B illustrates variations in an image value obtained in accordance with UBP.

FIG. 4 illustrates an image obtained as a result of performing a process according to a comparative example and the present invention.

FIG. 5 is a block diagram illustrating a photoacoustic apparatus according to an embodiment.

FIG. 6A schematically illustrates a probe according to an embodiment.

FIG. 6B schematically illustrates the probe according to the embodiment.

FIG. 7 is a block diagram illustrating the configuration of a computer and its surroundings according to an embodiment.

FIG. 8 is a flowchart of an image generating method according to an embodiment.

FIG. 9 is a flowchart of a process for generating image data in accordance with an embodiment.

FIG. 10A illustrates a histogram of an image value group according to an embodiment.

FIG. 10B illustrates a histogram of the image value group according to the embodiment.

FIG. 11A illustrates a characteristic information image obtained by the photoacoustic apparatus according to the embodiment.

FIG. 11B illustrates a characteristic information image obtained by the photoacoustic apparatus according to the embodiment.

FIG. 11C illustrates a characteristic information image obtained by the photoacoustic apparatus according to the embodiment.

### DESCRIPTION OF EMBODIMENTS

Embodiments of the present invention will be described below with reference to the drawings. The dimensions, materials, shapes, and relative positions of components to be described below may be changed, where appropriate, depending on the configuration of an apparatus to which the invention is applied or various conditions, and the scope of the invention should not be limited to the following description.

The present invention relates to generation of image data expressing a two-dimensional or three-dimensional spatial distribution and derived from a photoacoustic wave generated as a result of light irradiation. Photoacoustic image data expresses a spatial distribution of subject information of at least one of the generated sound pressure (initial sound pressure) of a photoacoustic wave, an optical absorption energy density, an optical absorption coefficient, and the concentration (such as an oxygen saturation) of a material constituting a subject.

A biological organism serving as a main subject in photoacoustic imaging has light scattering and absorbing properties. Therefore, as light advances deep into the biological organism, the light intensity exponentially attenuates. As a result, a photoacoustic wave with a large amplitude tends to occur near the surface of the subject, whereas a photoacoustic wave with a small amplitude tends to occur in a deep area of the subject. In particular, a photoacoustic wave with a large amplitude tends to occur from a blood vessel existing near the surface of the subject.

In a reconfiguration method called universal back-projection (UBP) described in NPL 1, a reception signal is back-projected onto a circular arc centered on a transducer. In this case, a reception signal of a photoacoustic wave with a large amplitude near the surface of the subject is back-projected onto a deep area of the subject, resulting in an artifact in the deep area of the subject. Therefore, when biological tissue existing in the deep area of the subject is to be formed into an image, the image quality (such as the contrast) may possibly deteriorate due to the artifact caused by the photoacoustic wave generated from the surface of the subject. Accordingly, it may sometimes be difficult to differentiate whether or not an image within the image is an image of a target (observation target).

The present invention facilitates the differentiation of whether or not a target (observation target) exists at a certain position within an image. Specifically, the present invention facilitates the differentiation of whether the possibility of the existence of a target at a certain position within an image is high or low. In this description, the determination of whether or not a target exists corresponds to the determination of whether the possibility of the existence of a target is high or low. A process according to the present invention will be described below.

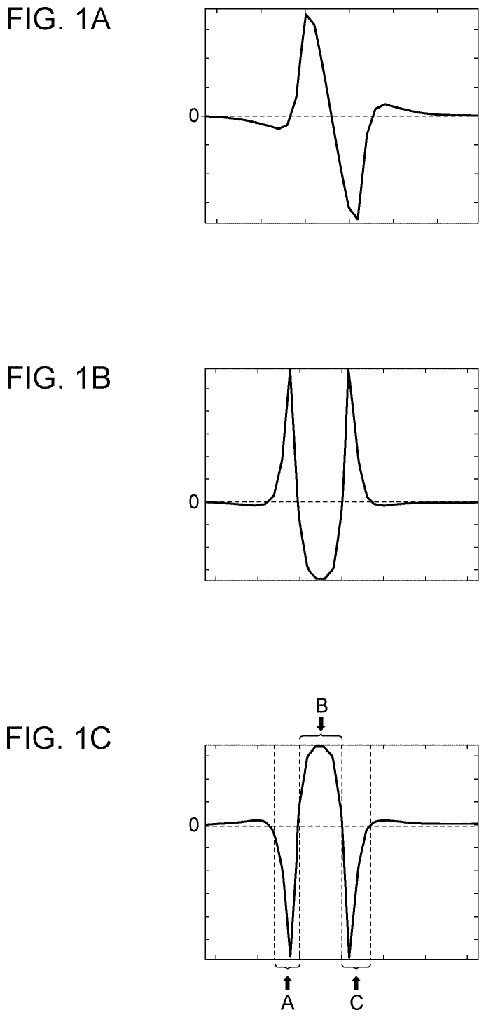

A reception signal of a photoacoustic wave is known to normally have a waveform called an N-shape as shown in FIG. 1A. In UBP, a time differentiation process is performed on an N-shaped signal shown in FIG. 1A, so that a time derivative signal shown in FIG. 1B is generated. Subsequently, a positive-negative inversion process is performed for inverting the positive and negative signal levels of the time derivative signal, so that a positive-negative-inverted signal shown in FIG. 1C is generated. The signal (also called a projection signal) generated as a result of performing the time differentiation process and the positive-negative inversion process on the N-shaped signal has parts with negative values, as indicated by arrows A and C in FIG. 1C, and a part with a positive value, as indicated by an arrow B in FIG. 1C.

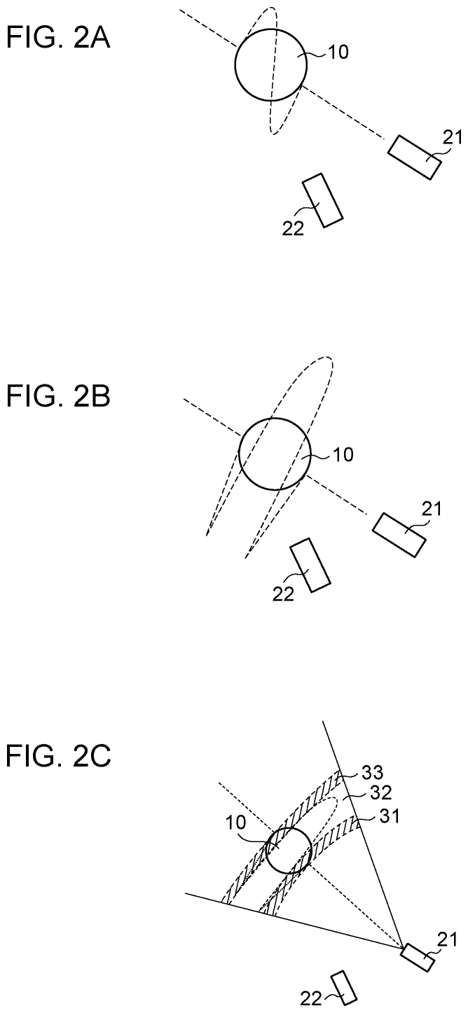

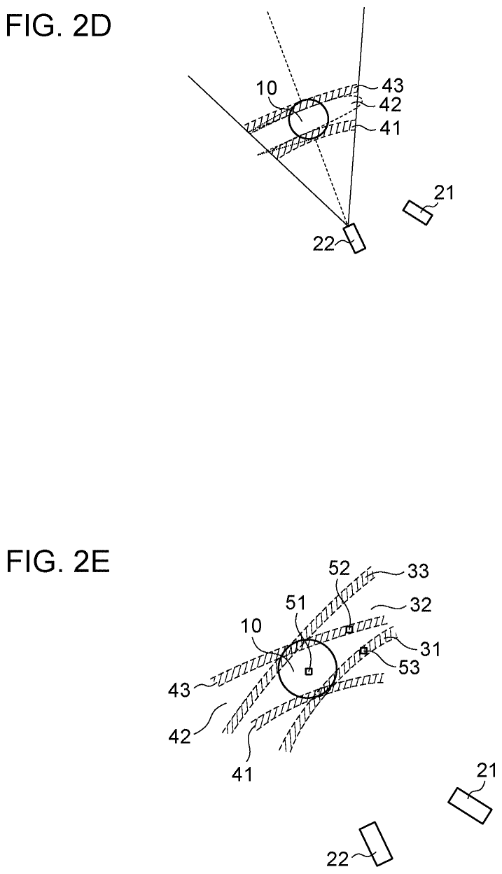

FIGS. 2A to 2E illustrate an example where UBP is applied to a case where a photoacoustic wave generated from a target 10, which is a micro-spherical light absorber, within a subject is received by a transducer 21 and a transducer 22. When light is radiated onto the target 10, a photoacoustic wave is generated, and the photoacoustic wave is sampled as an N-shaped signal by the transducer 21 and the transducer 22. FIG. 2A illustrates a state where the N-shaped reception signal sampled by the transducer 21 is superposed on the target 10. Although only the reception signal output from the transducer 21 is shown for the sake of convenience, a reception signal is similarly output from the transducer 22.

FIG. 2B illustrates a state where a projection signal obtained by performing a time differentiation process and a positive-negative inversion process on the N-shaped reception signal shown in FIG. 2A is superposed on the target 10.

FIG. 2C illustrates a state where the projection signal obtained by using the transducer 21 is back-projected in accordance with UBP. In UBP, the projection signal is protected onto a circular arc centered on the transducer 21. In this case, the projection signal is back-projected onto a directional-angle range (e.g., 60°) of the transducer 21. As a result, the image indicates as if the target 10 exists across regions 31, 32, and 33. Each of the regions 31 and 33 is a region having a negative value, and the region 32 is a region having a positive value. In FIG. 2C, the regions 31 and 33 having the negative values are shaded in gray.

FIG. 2D illustrates a case where the projection signal obtained by using the transducer 22 is back-projected in accordance with UBP. As a result, the image indicates as if the target 10 exists across regions 41, 42, and 43. Each of the regions 41 and 43 is a region having a negative value, and the region 42 is a region having a positive value. In FIG. 2D, the regions 41 and 43 having the negative values are shaded in gray.

FIG. 2E illustrates a case where the projection signals corresponding to the plurality of transducers 21 and 22 are back-projected in accordance with UBP. The plurality of projection signals back-projected in this manner are combined, so that photoacoustic image data is generated.

As shown in FIG. 2E, at a position 51 within the target 10, the positive-value region 32 of the projection signal corresponding to the transducer 21 and the positive-value region 42 of the projection signal corresponding to the transducer 22 overlap. Specifically, in a region where the target 10 typically exists (also called a target region), positive-value regions dominantly overlap each other. Therefore, in a region where the target 10 exists, image data for each radiated light beam normally tends to have a positive value.

On the other hand, at a position 52 outside the target 10, the positive-value region 32 of the projection signal corresponding to the transducer 21 and the negative-value region 43 of the projection signal corresponding to the transducer 22 overlap. At a position 53 outside the target 10, the negative-value region 31 of the projection signal corresponding to the transducer 21 and the positive-value region 41 of the projection signal corresponding to the transducer 22 overlap. Accordingly, in a region outside the target 10, positive-value regions and negative-value regions tend to complexly overlap. Specifically, in a region outside the target 10, there is a tendency in which image data for each radiated light beam may have a positive value or a negative value. A conceivable reason for such a tendency is a change in the relative position between the transducer 22 and the target 10 for each radiated light beam.

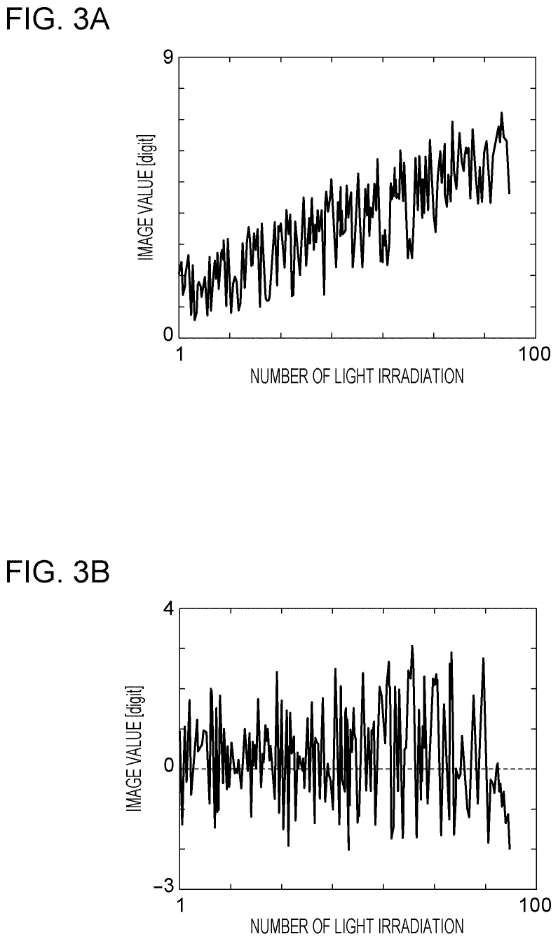

Next, the following description relates to variations in the value of image data (image value) for each radiated light beam when the combination of photoacoustic-wave reception positions is changed for every radiated light beam. FIG. 3A illustrates variations in the value of the image data (image value) when the region of the target 10 is reconfigured in accordance with UBP described in NPL 1. The abscissa axis indicates the number of light irradiation, and the ordinate axis indicates the image value. FIG. 3B illustrates variations in the value of the image data (image value) when a region outside the target 10 is reconfigured in accordance with UBP' described in NPL 1. The abscissa axis indicates the number of light irradiation, and the ordinate axis indicates the image value.

It is apparent from FIG. 3A that the image value of the region of the target 10 varies according to each radiated light beam but is always a positive value. On the other hand, it is comprehensible from FIG. 3B that the image value of the region outside the target 10 may either be a positive value or a negative value for each radiated light beam.

When image data is generated by combining pieces of image data corresponding to all radiated light beams, the ultimate image value becomes large since positive values are combined in the region of the target 10. In contrast, in the region outside the target 10, positive and negative values of the image data cancel out each other, so that the ultimate image value becomes smaller than that in the region of the target 10. As a result, the existence of the target 10 can be visually recognized in the image based on the photoacoustic image data.

However, in the region outside the target 10, the image value does not become zero regardless of the fact that a target does not exist, sometimes resulting in the ultimate image value being a positive value. In this case, an artifact occurs at a position outside the target 10, thus lowering the visibility of the target.

It is desirable to facilitate the differentiation of whether an image in a certain region is an image of a target or an image of an object other than the target.

In order to solve the aforementioned problem, the present inventor has focused on the fact that the region of the target and the region outside the target typically have different tendencies with respect to the variation characteristics of the image value of the image data for each radiated light beam. Specifically, the present inventor has conceived a method of differentiating the region of the target and the region outside the target from each other from the variation characteristics of the image value of the image data for each radiated light beam. The use of this method allows for accurate differentiation of a target.

Moreover, the present inventor has conceived an idea of displaying an image expressing a determination result indicating whether or not a region is a target region. By displaying such an image, it is easily differentiable whether or not a target exists at a certain position within the image.

Moreover, the present inventor has conceived an idea of setting a region of a target based on the above-described method and selectively extracting an image of the target from image data. Specifically, the present inventor has conceived an idea in which, if a target does not exist at a certain position, an image based on image data at the position is displayed with a brightness lower than the brightness corresponding to the image value at the position. According to such an image generating method, an image with the target in a highlighted state can be provided to a user. By displaying such an image, a user can easily differentiate whether or not a target exists at a certain position.

Furthermore, the present inventor has conceived an idea of displaying an image based on characteristic information indicating the characteristics of a plurality of pieces of image data corresponding to a plurality of times light is radiated. By displaying such characteristic information, a user can easily differentiate whether or not a target exists at a certain position.

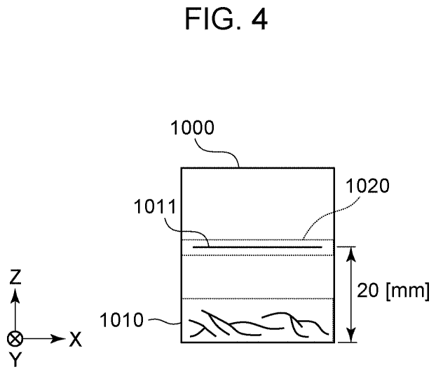

The details of a process according to the present invention will be described below in the following embodiment. The following embodiment to be described below relates to an example where a reception signal of a photoacoustic wave is generated in accordance with a simulation performed on a subject model 1000 shown in FIG. 4, and image data is generated by using the reception signal. FIG. 4 illustrates the subject model 1000 used in the simulation. A model created as the subject model 1000 has blood vessels 1010 existing near the surface and a 0.2 [mm] blood vessel 1011 extending in a Y-axis direction at a depth of 20 [mm] from the surface. In this subject model 1000, blood vessels are targets. A reception signal to be received by a receiver provided below the subject model 1000 in the plane of the drawing is created by simulating a photoacoustic wave generated from the blood vessels 1010 and 1011 within the subject model 1000 when light is radiated a plurality of times. The reception signal is created by changing the reception position of the photoacoustic wave for each radiated light beam in the simulation. Furthermore, a reconfiguration process is performed based on universal back-projection (UBP), to be described below, by using the reception signal obtained in accordance with the simulation, and image data corresponding to each radiated light beam is created.

This embodiment relates to an example where a photoacoustic apparatus generates photoacoustic image data. The configuration of the photoacoustic apparatus according to this embodiment and an information processing method will be described below.

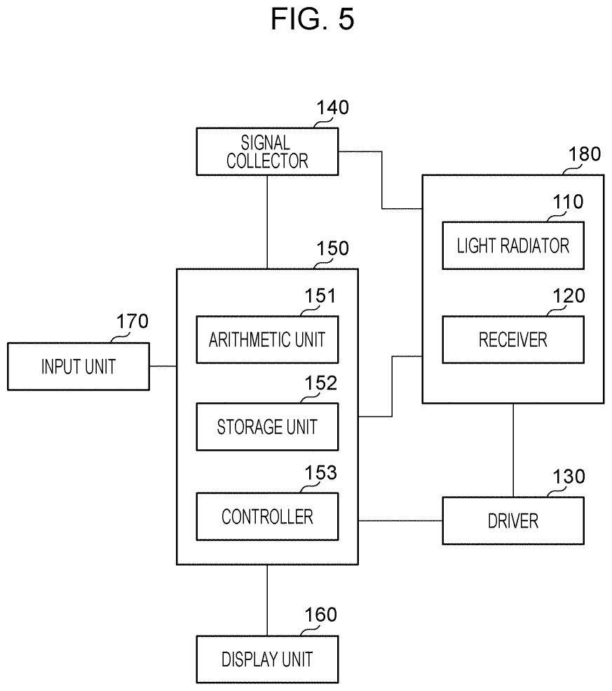

The configuration of the photoacoustic apparatus according to this embodiment will be described with reference to FIG. 5. FIG. 5 is a schematic block diagram of the entire photoacoustic apparatus. The photoacoustic apparatus according to this embodiment has a probe 180 including a light radiator 110 and a receiver 120, a driver 130, a signal collector 140, a computer 150, a display unit 160, and an input unit 170.

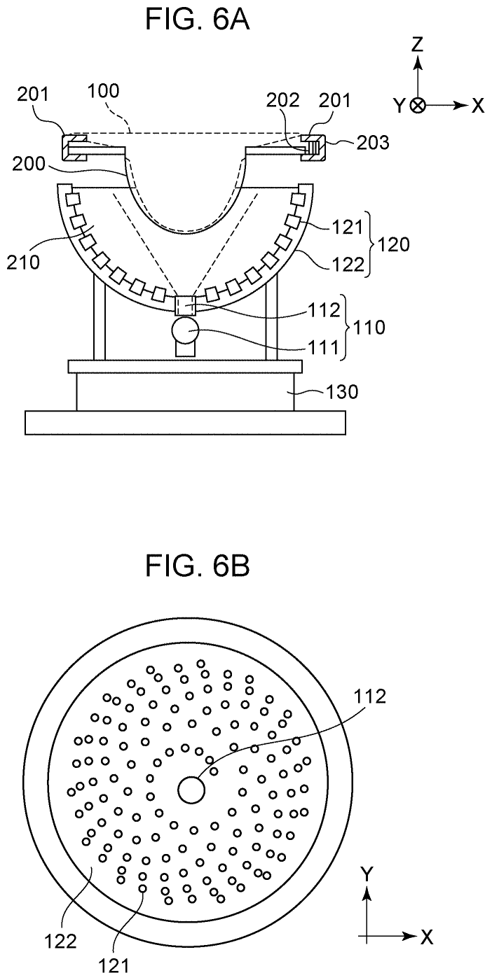

FIG. 6 schematically illustrates the probe 180 according to this embodiment. A target to be measured is a subject 100. The driver 130 drives the light radiator 110 and the receiver 120 to perform mechanical scanning. The light radiator 110 radiates light onto the subject 100, so that an acoustic wave is generated within the subject 100. An acoustic wave derived from light and generated by a photoacoustic effect is also called a photoacoustic wave. The receiver 120 receives the photoacoustic wave so as to output an electric signal (photoacoustic signal) as an analog signal.

The signal collector 140 converts the analog signal output from the receiver 120 into a digital signal and outputs the digital signal to the computer 150. The computer 150 stores the digital signal output from the signal collector 140 as signal data derived from the photoacoustic wave.

The computer 150 performs signal processing on the stored digital signal so as to generate photoacoustic image data expressing a two-dimensional or three-dimensional spatial distribution of information related to the subject 100 (subject information). Moreover, the computer 150 causes the display unit 160 to display an image based on the obtained image data. A doctor who is a user can perform a diagnosis by checking the image displayed on the display unit 160. The display image is stored in a memory in the computer 150 or in a memory in a data management system connected to a modality device via a network based on a store command from the user or the computer 150.

The computer 150 also performs drive control on the components included in the photoacoustic apparatus. The display unit 160 may also display a graphical user interface (GUI) in addition to the image generated by the computer 150. The input unit 170 is configured such that the user can input information thereto. The user may use the input unit 170 to perform an operation for starting or terminating a measuring process or for giving a command for storing the created image.

The components of the photoacoustic apparatus according to this embodiment will be described in detail below.

### (Light Radiator 110)

The light radiator 110 includes a light source 111 that emits light and an optical system 112 that guides the light output from the light source 111 to the subject 100. The light includes pulsed light having a so-called rectangular wave or triangular wave.

The pulse width of the light emitted by the light source 111 may range between 1 ns and 100 ns inclusive. The wavelength of the light may range from about 400 nm to 1600 nm. In a case where a blood vessel is to be imaged with high resolution, the light used may have a wavelength with high absorbability (between 400 nm and 700 nm inclusive) in the blood vessel. In a case where a deep area of a biological organism is to be imaged, the light used may have a wavelength with typically low absorbability (between 700 nm and 1100 nm inclusive) in background tissue (water and fat) of the biological organism.

The light source 111 used may be a laser or a light-emitting diode. When the measuring process is to be performed by using light with a plurality of wavelengths, a wavelength-variable light source may be used. If a plurality of wavelengths are to be radiated onto the subject, a plurality of light sources that generate light beams having different wavelengths may be prepared, and the beams may be alternately radiated from the respective sources. If a plurality of light sources are used, they are collectively expressed as a light source. A laser used may be any of various lasers, such as a solid-state laser, a gas laser, a dye laser, or a semiconductor laser. For example, a pulsed laser, such as a neodymium-doped yttrium aluminum garnet (Nd:YAG) laser or an alexandrite laser, may be used as the light source. Alternatively, a titanium-sapphire (Ti:sa) laser or optical parametric oscillator (OPO) laser using Nd:YAG laser light as excitation light may be used as the light source. As another alternative, a flash lamp or a light-emitting diode may be used as the light source 111. As another alternative, a microwave source may be used as the light source 111.

The optical system 112 used may include optical elements, such as a lens, a mirror, a prism, an optical fiber, a diffuser, and a shutter.

The permissible intensity of light that can be radiated onto biological tissue is set to a maximum permissible exposure (MPE) in accordance with a safety standard indicated below (such as IEC 60825-1: Safety of laser products, JIS C 6802: Safety of laser products, FDA: 21CFR Part 1040.10, or ANSI Z136.1: Laser Safety Standards). A maximum permissible exposure prescribes the intensity of h that can be radiated per unit area. Therefore, light collectively radiated over a wide area of the surface of a subject E, so that a large amount of light can be guided to the subject E, whereby a photoacoustic wave can be received with a high signal-to-noise (S/N) ratio. In a case where the subject 100 is biological tissue of, for example, a breast, the light emitter of the optical system 112 may be constituted by, for example, a diffuser that diffuses light so that high-energy light with an increased beam diameter can be radiated. On the other hand, in a photoacoustic microscope, the light emitter of the optical system 112 may be constituted by, for example, a lens to increase the resolution, so that a beam can be radiated in a focused state.

Instead of being equipped with the optical system 112, the light radiator 110 may radiate light directly onto the subject 100 from the light source 111.

### (Receiver 120)

The receiver 120 includes a transducer 121 that outputs an electric signal by receiving an acoustic wave, and also includes a supporter 122 that supports the transducer 121. The transducer 121 may also serve as a transmitter that transmits an acoustic wave. A transducer serving as a receiver and a transducer serving as a transmitter may be a single (common) transducer or may be separate from each other.

The transducer 121 may be composed of, for example, a piezoelectric ceramic material as typified by lead zirconate titanate (PZT) or a high-polymer piezoelectric film material as typified by polyvinylidene fluoride (PVDF). Alternatively, an element other than a piezoelectric element may be used. For example, a capacitive micro-machined ultrasonic transducer (CMUT) or a transducer that uses a Fabry-Perot interferometer may be used. Any transducer may be employed so long as it can output an electric signal by receiving an acoustic wave. A signal that can be obtained by a transducer is a time-resolved signal. Specifically, the amplitude of a signal obtained by a transducer indicates a value based on a sound pressure received by the transducer at each time point (e.g., a value proportional to the sound pressure).

Frequency components constituting a photoacoustic wave typically range from 100 KHz to 100 MHz, and the transducer 121 that can be employed is capable of detecting these frequencies.

The supporter 122 may be composed of, for example, a metallic material with high mechanical strength. In order to cause a large amount of radiated light to enter a subject, the surface of the supporter 122 closer toward the subject 100 may be processed to be a mirror-finished surface or a light-scattering surface. In this embodiment, the supporter 122 has the shape of a hemispherical shell and can support a plurality of transducers 121 on the hemispherical shell. In this case, the directional axes of the transducers 121 disposed on the supporter 122 converge near the center of curvature of the hemisphere. When an image is formed by using signals output from the plurality of transducers 121, the image quality near the center of curvature becomes high The supporter 122 may have any configuration so long as it can support the transducers 121. On the supporter 122, the plurality of transducers may be arranged in a flat surface or curved surface called a one-dimensional array, a 1.5-dimensional array, a 1.75-dimensional array, or a 2-dimensional array. The plurality of transducers 121 correspond to a plurality of receivers.

The supporter 122 may function as a container that stores an acoustic matching material 210. Specifically, the supporter 122 may serve as a container for disposing the acoustic matching material 210 between the transducers 121 and the subject 100.

The receiver 120 may include an amplifier that amplifies time-series analog signals output from the transducers 121. Moreover, the receiver 120 may include an analog-to-digital (A/D) converter that converts the time-series analog signals output from the transducers 121 into time-series digital signals. Specifically, the receiver 120 may include the signal collector 140 to be described later.

In order to detect an acoustic wave at various angles, the transducers 121 may ideally be disposed to entirely surround the subject 100. However, if the transducers cannot be disposed to entirely surround the subject 100 due to the subject 100 being large, a state similar thereto may be achieved by disposing the transducers on the hemispherical supporter 122 in an entirely surrounding fashion.

The arrangement and the number of transducers and the shape of the supporter may be optimized in accordance with the subject. With regard to the present invention, any type of receiver 120 may be employed.

The space between the receiver 120 and the subject 100 is filled with a medium that allows a photoacoustic wave to propagate therethrough. The medium used is a material that allows an acoustic wave to propagate therethrough, has matching acoustic characteristics at the interface between the subject 100 and the transducers 121, and has high transmittance for a photoacoustic wave as much as possible. For example, water or ultrasound gel may be used as this medium.

FIG. 6A is a side view of the probe 180, and FIG. 6B is a top view of the probe 180 (as viewed from above the plane of drawing in FIG. 6A). The probe 180 according to this embodiment shown in FIGS. 6A and 6B has the receiver 120 in which the plurality of transducers 121 are three-dimensionally disposed on the hemispherical supporter 122 having an opening. Furthermore, in the probe 180 shown in FIGS. 6A and 6B, the light emitter of the optical system 112 is disposed at the bottom of the supporter 122.

As shown in FIGS. 6A and 6B, in this embodiment, the subject 100 comes into contact with a retainer 200 so that the shape of the subject 100 is maintained. In this embodiment, if the subject 100 is a breast, a bed that supports the subject person in a prone position is provided with an opening for fitting the breast therein, so that the breast fitted in the vertical direction through the opening can be measured.

The space between the receiver 120 and the retainer 200 is filled with a medium (acoustic matching material 210) that allows a photoacoustic wave to propagate therethrough. The medium used is a material that allows a photoacoustic wave to propagate therethrough, has matching acoustic characteristics at the interface between the subject 100 and the transducers 121, and has high transmittance for a photoacoustic wave as much as possible. For example, water, castor oil, or ultrasound gel may be used as this medium.

The retainer 200 as a retaining unit is used for maintaining the shape of the subject 100 during a measuring process. By using the retainer 200 to retain the subject 100, movement of the subject 100 can be suppressed and the subject 100 can be positionally kept within the retainer 200. The retainer 200 may be composed of a resin material, such as polycarbonate, polyethylene, or polyethylene terephthalate.

The retainer 200 is preferably composed of a material having enough hardness for retaining the subject 100. The retainer 200 may be composed of a material that allows light used in the measuring process to be transmitted therethrough. The retainer 200 may be composed of a material with an impedance that is about the same as that of the subject 100. If the subject 100 has a curved surface of, for example, a breast, the retainer 200 may be shaped to have a recessed shape. In this case, the subject 100 can be fitted in the recess of the retainer 200.

The retainer 200 is attached to an attachment section 201. The attachment section 201 may be configured such that a plurality of types of retainers 200 are replaceable in conformity with the size of the subject. For example, the attachment section 201 may be configured to allow for replacement of retainers with various radii of curvature and various centers of curvature.

The retainer 200 may have a tag 202 with information about the retainer 200 registered therein. Examples of the information that can be registered in the tag 202 include the radius of curvature and the center of curvature of the retainer 200, the sound velocity, and an identification ID. The information registered in the tag 202 is read by a reader 203 and is forwarded to the computer 150. In order to easily read the tag 202 when the retainer 200 is attached to the attachment section 201, the reader 203 may be set in the attachment section 201. For example, the tag 202 is a bar code, and the reader 203 is a bar code reader.

### (Driver 130)

The driver 130 changes the relative position between the subject 100 and the receiver 120. In this embodiment, the driver 130 is a device that moves the supporter 122 in the XY direction and is an electric XY stage equipped with a stepping motor. The driver 130 includes a motor, such as a stepping motor, for generating a driving force, a driving mechanism that transmits the driving force, and a position sensor that detects positional information of the receiver 120. The driving mechanism used may be, for example, a lead screw mechanism, a link mechanism, a gear mechanism, or a hydraulic mechanism. The position sensor used may be, for example, a potentiometer that uses an encoder, a variable resistor, a linear scale, a magnetic sensor, an infrared sensor, or an ultrasonic sensor.

The driver 130 is not limited to the type that changes the relative position between the subject 100 and the receiver 120 in the XY (two-dimensional) direction, and may be changed to a type that changes the relative position one-dimensionally or three-dimensionally. With regard to the movement path, scanning may be performed planarly in a spiral pattern or in a line-and-space fashion, or the movement path may be inclined to three-dimensionally conform to the surface of the body. The probe 180 may be moved so as to maintain a fixed distance from the surface of the subject 100. In this case, the driver 130 may measure the amount of movement of the probe by, for example, monitoring the rotation speed of the motor.

The driver 130 may fix the receiver 120 and move the subject 100 so long as the relative position between the subject 100 and the receiver 120 can be changed. If the subject 100 is to be moved, a configuration that moves the subject 100 by moving the retainer that retains the subject 100 is conceivable. Alternatively, the subject 100 and the receiver 120 may both be moved.

The driver 130 may move the relative position continuously or may move the relative position in a step-and-repeat fashion. The driver 130 may be an electric stage that moves the relative position along a programmed path, or may be a manual stage. Specifically, the photoacoustic apparatus may be not equipped with the driver 130 and may be of a handheld type in which the user holds and operates the probe 180.

In this embodiment, the driver 130 performs scanning by simultaneously driving the light radiator 110 and the receiver 120. Alternatively, the driver 130 may drive the light radiator 110 alone or may drive the receiver 120 alone.

### (Signal Collector 140)

The signal collector 140 includes an amplifier that amplifies electric signals, which are analog signals, output from the transducers 121, and also includes an A/D converter that converts the analog signals output from the amplifier into digital signals. The signal collector 140 may be constituted by, for example, a field programmable gate array (FPGA) chip. The digital signals output from the signal collector 140 are stored in a storage unit 152 within the computer 150. The signal collector 140 is also called a data acquisition system (DAS). The concept of an electric signal in this description is that the electric signal includes both an analog signal and a digital signal. Alternatively, an optical sensor, such as a photodiode, may detect light output from the light radiator 110, and the signal collector 140 may start the above-described process in synchronization with this detection result. As another alternative, the signal collector 140 may start the above-described process in synchronization with a command given by using, for example, a freeze button.

### (Computer 150)

The computer 150 as a display controller includes an arithmetic unit 151, the storage unit 152, and a controller 153. The functions of the respective components will be described in the description of the processing flow.

Units having the role of an arithmetic function and serving as the arithmetic unit 151 may be constituted by a processor, such as a central processing unit (CPU) or a graphics processing unit (CPU), and an arithmetic circuit, such as an FPGA chip. These units may be constituted by a single processor and arithmetic circuit or by a plurality of processors and arithmetic circuits. The arithmetic unit 151 may receive various types of parameters, such as the sound velocity of the subject and the configuration of the retainer, from the input unit 170 and may process a reception signal.

The storage unit 152 may be constituted by a non-transitory storage medium, such as a read-only memory (ROM), a magnetic disk, or a flash memory. Moreover, the storage unit 152 may be a volatile medium, such as a random access memory (RAM). A storage medium that stores a program is a non-transitory storage medium. The storage unit 152 may be constituted by a single storage medium or by a plurality of storage media.

The storage unit 152 can store image data indicating a photoacoustic image generated by the arithmetic unit 151 in accordance with a method to be described below.

The controller 153 is constituted by an arithmetic element, such as a CPU. The controller 153 controls the operation of each component of the photoacoustic apparatus. The controller 153 may receive command signals given by various types of operations, such as a command for starting a measuring process, from the input unit 170 so as to control the components of the photoacoustic apparatus. Furthermore, the controller 153 reads a program code stored in the storage unit 152 and controls the operation of each component of the photoacoustic apparatus. For example, the controller 153 may control the light emission timing of the light source 111 via a control line. If the optical system 112 includes a shutter, the controller 153 may control the opening and closing of the shutter via a control line.

The computer 150 may be a dedicatedly-designed workstation. The components of the computer 150 may be constituted by different hardware units. Moreover, at least one of the components of the computer 150 may be constituted by a single hardware unit.

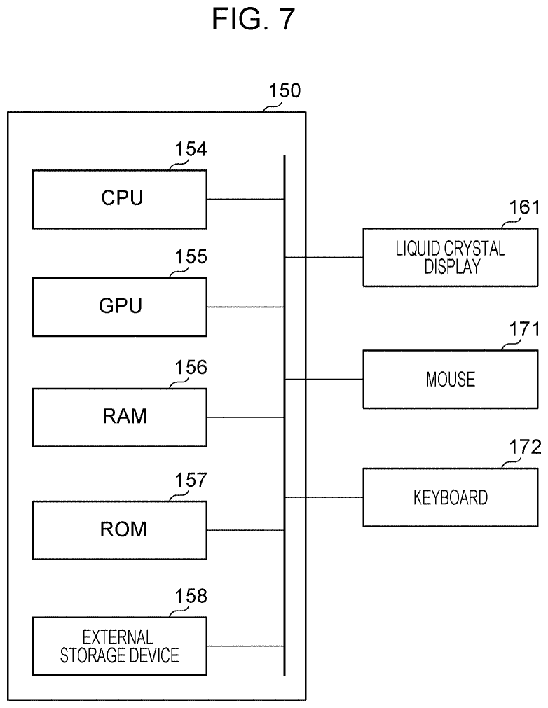

FIG. 7 illustrates a specific configuration example of the computer 150 according to this embodiment. The computer 150 according to this embodiment is constituted by a CPU 154, a GPU 155, a RAM 156, a ROM 157, and an external storage device 158. The computer 150 is connected to a liquid crystal display 161 as the display unit 160 and to a mouse 171 and keyboard 172 as the input unit 170.

The computer 150 and the plurality of transducers 121 may be provided by being accommodated within a common housing. Alternatively, some signal processing may be performed in the computer accommodated in the housing, while the remaining signal processing may be performed in a computer provided outside the housing. In this case, the computers provided inside and outside the housing may collectively be referred to as a computer according to this embodiment. In other words, the hardware constituting the computer does not have to be accommodated within a single housing.

### (Display Unit 160)

The display unit 160 is a display, such as a liquid crystal display, an organic electroluminescence (EL) field emission display (FED), an eyeglasses-type display, or a head mount display, and displays an image based on volume data obtained by the computer 150 or a numerical value of a specific position. The display unit 160 may also display a GUI used for displaying an image based on volume data or for operating a device. When subject information is to be displayed, the subject information may be displayed after image processing (such as an adjustment of a brightness value) is performed in the display unit 160 or the computer 150. The display unit 160 may be provided separately from the photoacoustic apparatus. The computer 150 can transmit photoacoustic image data to the display unit 160 in a wired or wireless manner.

### (Input Unit 170)

The input unit 170 used may be a control console operable by a user and constituted by, for example, a mouse and a keyboard. Alternatively, the display unit 160 may be constituted by a touchscreen, such that the display unit 160 may be used as the input unit 170.

The input unit 170 may be configured to receive information about the position and the depth to be observed. The input method may involve inputting a numerical value or inputting information by operating a slider bar. Furthermore, the image displayed on the display unit 160 may be updated in accordance with the input information. Accordingly, a user can set an appropriate parameter while checking an image generated in accordance with the parameter set by the user.

Furthermore, a user may operate the input unit 170 provided remotely from the photoacoustic apparatus and may transmit information input by using the input unit 170 to the photoacoustic apparatus via a network.

The components of the photoacoustic apparatus may be constituted by separate devices, or may be constituted by a single integrated device. Furthermore, at least one of the components of the photoacoustic apparatus may be constituted by a single integrated device.

Furthermore, information to be exchanged between the components of the photoacoustic apparatus may be exchanged in a wired or wireless manner.

### (Subject 100)

The subject 100 does not constitute the photoacoustic apparatus, but will be described below. The photoacoustic apparatus according to this embodiment can be used for the purpose of diagnosing or monitoring a chemical treatment of, for example, a malignant tumor or a blood vessel disease in a human or an animal. Therefore, it is assumed that the subject 100 is a target diagnostic site, such as a breast, an organ, a vascular network, a head region, a neck region, an abdominal region, and extremities including fingers and toes of a biological organism, specifically, a human or an animal. For example, if the target to be measured is a human, the light absorber may be, for example, oxyhemoglobin or deoxyhemoglobin, a blood vessel containing a large amount thereof, or a new blood vessel formed near a tumor. Alternatively, the light absorber may be, for example, a plaque on a carotid wall. As another alternative, the light absorber may be, for example, melanin contained in the skin, collagen, or lipid. Furthermore, the light absorber may be a pigment, such as methylene blue (MB) or indocyanine green (ICG), fine gold particles, or an externally introduced material obtained by collecting or chemically modifying the pigment and the fine gold particles. Moreover, the subject 100 may be a phantom that imitates a biological organism.

In this description, a light absorber subjected to imaging described above is called a target. A light absorber not subjected to imaging, that is, not subjected to observation, is not a target. For example, if the subject is a breast and the light absorber of the target is a blood vessel, it is conceivable that the tissue constituting the breast, such as fat and mammary gland, not a target. If the target is a blood vessel, it is conceivable that light with a wavelength suitable for light absorption in the blood vessel is used.

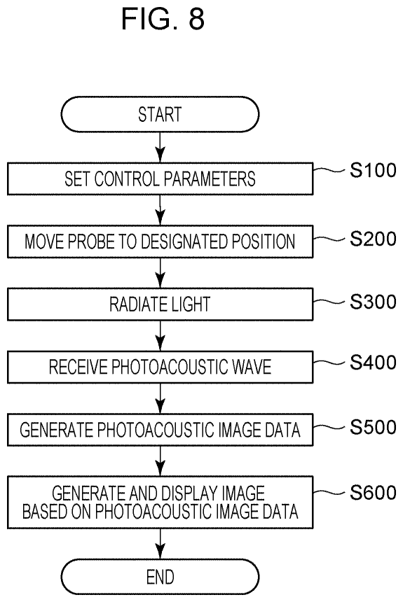

Next, an image display method including information processing according to this embodiment will be described with reference to FIG. 8. The steps are executed by the computer 150 controlling the operation of each component of the photoacoustic apparatus.

### (S100: Step for Setting Control Parameters)

The user uses the input unit 170 to designate control parameters, such as the irradiation conditions (e.g., cyclic frequency and wavelength) of the light radiator 110 necessary for acquiring subject information and the position of the probe 180. The computer 150 sets the control parameters set based on the command by the user.

### (S200: Step for Moving Probe to Designated Position)

Based on the control parameters designated in step S100, the controller 153 causes the driver 130 to move the probe 180 to the designated position. If imaging at a plurality of positions is designated in step S100, the driver 130 first moves the probe 180 to a first designated position. The driver 130 may move the probe 180 to a preliminarily-programmed position when a measurement start command is given. In a case of a hand-held type, the user may hold the probe 180 and move the probe 180 to a desired position.

### (S300: Step for Radiating Light)

The light radiator 110 radiates light onto the subject 100 based on the control parameters designated in step S100.

Light generated from the light source 111 is radiated onto the subject 100 as pulsed light via the optical system 112. Then, the pulsed light is absorbed within the subject 100, and a photoacoustic wave occurs due to a photoacoustic effect. In addition to transmitting the pulsed light, the light radiator 110 transmits a synchronization signal to the signal collector 140.

### (S400: Step for Receiving Photoacoustic Wave)

When the signal collector 140 receives the synchronization signal transmitted from the light radiator 110, the signal collector 140 starts to perform signal collecting operation. Specifically, the signal collector 140 performs amplification and A/D conversion on an analog electric signal output from the receiver 120 and derived from an acoustic wave, so as to generate an amplified digital electric signal, and outputs the signal to the computer 150. The computer 150 stores the signal transmitted from the signal collector 140 into the storage unit 152. If imaging at a plurality of scan positions is designated in step S100, steps S200 to S400 are repeated at each of the designated scan positions, thereby repeating the process of radiating pulsed light and generating a digital signal derived from an acoustic wave. Alternatively, when light is emitted, the computer 150 may acquire and store positional information of the receiver 120 at the time of light emission based on an output from the position sensor of the driver 130.

### (S500: Step for Generating Photoacoustic Image Data)

The arithmetic unit 151 of the computer 150 serving as an image data generator generates photoacoustic image data based on signal data stored in the storage unit 152 and stores the photoacoustic image data in the storage unit 152.

A reconfiguration algorithm used for converting signal data into volume data as a spatial distribution may be an analytic reconfiguration method, such as a time-domain-based back-projection method or a Fourier-domain-based back-projection method, or a model-based method (repetitive calculation method). Examples of the time-domain-based back-projection method include the universal back-projection (UBP) method, the filtered back-projection (FBP) method, and the delay-and-sum method.

The arithmetic unit 151 may calculate a light fluence distribution within the subject 100 when light is radiated onto the subject 100, and may divide an initial sound pressure distribution by the light fluence distribution so as to acquire absorption-coefficient-distribution information. In this case, the absorption-coefficient-distribution information may be acquired as photoacoustic image data. The computer 150 can calculate a light-fluence spatial distribution within the subject 100 in accordance with a method of numerically solving a transport equation or a diffusion equation expressing the behavior of light energy in a medium that absorbs and scatters light. The numerically solving method used may be, for example, the finite element method, the finite difference method, or the Monte Carlo method. For example, the computer 150 may solve a light diffusion equation indicated in expression (1) so as to calculate a light-fluence spatial distribution within the subject 100.

$$\frac{1}{c} \frac{\partial }{\partial t} Φ ( r , t ) = - {μ}_{a} Φ ( r , t ) + ∇ \cdot ( D ∇ Φ ( r , t ) ) + S ( r , t ) \qquad ( 1 )$$

In this case, D denotes a diffusion coefficient, μₐ denotes an absorption coefficient, S denotes an input intensity of radiated light, ϕ denotes an incoming light fluence, r denotes a position, and t denotes time.

Furthermore, step S300 and step S400 may be performed by using light beams of a plurality of wavelengths, and the arithmetic unit 151 may acquire absorption-coefficient-distribution information corresponding to each of the light beams of the plurality of wavelengths. Then, based on the absorption-coefficient-distribution information corresponding to each of the light beams of the plurality of wavelengths, the arithmetic unit 151 may acquire, as photoacoustic image data, spatial distribution information serving as spectral information and indicating the concentration of the material constituting the subject 100. Specifically, the arithmetic unit 151 may acquire spectral information by using signal data corresponding to the light beams of the plurality of wavelengths.

(S600: Step for Generating and Displaying image Based on Photoacoustic Image Data)

The computer 150 serving as a display controller generates an image based on the photoacoustic image data obtained in step S500 and causes the display unit 160 to display the image. An image value of the image data may directly be used as a brightness value of the display image. Alternatively, the brightness of the display image may be set by additionally performing predetermined processing on the image value of the image data. For example, if the image value is a positive value, the image value may be allocated to the brightness. If the image value is a negative value, a display image with a brightness of zero may be generated.

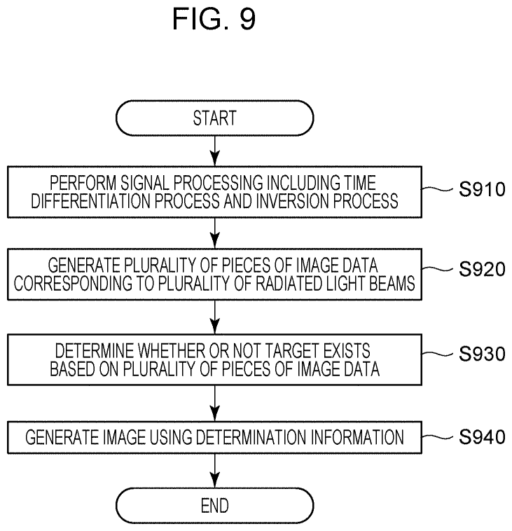

Next, a characteristic image generating method according to this embodiment will be described with reference to a flowchart of an image generating method shown in FIG. 9. With regard to an image-data generating method and an image display method based on image data in the flowchart shown in FIG. 9, the method performed in step S500 or S600 may be applied.

### (S910: Step for Performing Time Differentiation Process and Inversion Process on Reception Signal)

The computer 150 serving as a signal processor performs signal processing, including a time differentiation process and an inversion process for inverting positive and negative signal levels, on a reception signal stored in the storage unit 152. The reception signal having undergone such signal processing is also referred to as a projection signal. In this step, the signal processing is performed on each reception signal stored in the storage unit 152. As a result, projection signals respectively corresponding to the plurality of radiated light beams and the plurality of transducers 121 are generated.

For example, as indicated in expression (2), the computer 150 performs the time differentiation process and the inversion process (involving adding a negative sign to a time derivative signal) on a reception signal p(r, t) to generate a projection signal b(r, t), and stores the projection signal b(r, t) in the storage unit 152.

$$b ( r , t ) = 2 p ( r , t ) - 2 t \frac{\partial p ( r , t )}{\partial t} \qquad ( 2 )$$

In this case, r denotes a reception position, t denotes time elapsed from light irradiation, p(r, t) denotes a reception signal indicating the sound pressure of an acoustic wave received at the reception position r after the elapsed time t, and b(r, t) denotes a projection signal. In addition to the time differentiation process and the inversion process, other signal processing may be performed. Examples of other signal processing include at least one of frequency filtering (e.g., low pass, high pass, band pass), deconvolution, envelope detection, and wavelet filtering.

In this step, the inversion process does not necessarily have to be performed. Even in this case, the advantages of this embodiment are not impaired.

### (S920: Step for Generating Plurality of Pieces of Image Data)

The computer 150 serving as an image data generator generates a plurality of pieces of photoacoustic image data based on the reception signals (projection signals) generated in step S910 and respectively corresponding to the plurality of radiated light beams and the plurality of transducers 121. Photoacoustic image data may be generated for every radiated light beam, or a single piece of photoacoustic image data may be generated from a projection signal derived from a plurality of radiated light beams, so long as a plurality of pieces of photoacoustic image data can be generated.

For example, as indicated in expression (3), the computer 150 generates image data indicating a spatial distribution of an initial sound pressure p₀ for each radiated light beam based on a projection signal b(rᵢ, t). As a result, image data corresponding to each radiated light beam is generated, whereby a plurality of pieces of image data can be acquired.

$${p}_{0} ( {r}_{0} ) = \frac{{\sum }_{i}^{N} b ( {r}_{i} , t = \frac{\vert {r}_{i} - {r}_{0} \vert}{c} ) \cdot {ΔΩ}_{i}}{{\sum }_{i}^{N} {ΔΩ}_{i}} \qquad ( 3 )$$

In this case, r₀ denotes a position vector indicating a position where reconfiguration is to be performed (also called a reconfiguration position or a position of interest), p₀(r₀) denotes an initial sound pressure at the reconfiguration position, and c denotes a sound velocity on a propagation path. Furthermore, ΔΩᵢ denotes a solid angle from the reconfiguration position to an i-th transducer 121, and N denotes the number of transducers 121 to be used for the reconfiguration. Expression (3) indicates that phasing addition (back-protection) is performed by multiplying the projection signal by the weight of the solid angle.

As described above, in this embodiment, image data can be generated in accordance with a reconfiguration method, such as an analytic reconfiguration method or a model-based reconfiguration method.

### (S930: Step for Determining Whether or Not Target Exists Based on Plurality of Pieces of Image Data)

The computer 150 serving as a characteristic-information acquiring unit first analyses the variation characteristics of the image values of the plurality of pieces of image data acquired in step S920. The computer 150 acquires this analysis result as characteristic information indicating the characteristics of a data group (image value group) including values of the plurality of pieces of image data.

Then, based on characteristic information indicating the characteristics of an image value group at a certain position, the computer 150 serving as an information acquiring unit determines whether or not a target exists at the certain position and acquires determination information indicating the determination result. Specifically, the determination information indicates a possibility of the existence of a target at the certain position.

The characteristic information of the image value group at the certain position may be a statistic value including at least one of a median value, an average value, a standard deviation value, a variance value, entropy, and negentropy of the image value group at the certain position.

Specifically, this embodiment focuses on the characteristics (non-Gaussian characteristics) indicating whether or not the distribution of the image value group at the certain position is a normal distribution when a real image and an artifact are to be distinguished from each other. The term “non-Gaussian” indicates that the distribution of a certain data group has deviated from the normal distribution (Gaussian distribution). From the theory of probability, it is described that a distribution obtained by adding various independent random variables becomes more similar to the normal distribution (Gaussian distribution) in accordance with the central limit theorem. This is applied to, for example, noise superposed on a reception signal of a photoacoustic wave. Noise superposed on a reception signal of a photoacoustic wave contains noise, such as thermal noise, switching noise of a power source, and electromagnetic noise. In other words, noise superposed on a reception signal of a photoacoustic wave can be expressed as a sum of a plurality of independent random variables, such as a random variable of thermal noise, a random variable of switching noise of a power source, and a random variable of electromagnetic noise. For example, the signal collector 140 converts, that is, samples, an analog signal output from the receiver 120 into a digital signal at 100 [MHz]. In this case, a noise component existing in one sample of the reception signal sampled at 100 [MHz] contains a plurality of random variables. When the number of samples of the reception signal is further increased and the distribution is checked, the distribution becomes more similar to the normal distribution with increasing number of samples. This is an indication of the central limit theorem in the noise superposed on the reception signal of the photoacoustic wave.

Next, the distribution of a data group of image values of a plurality of pieces of photoacoustic image data will be examined from the viewpoint of central limit theorem.

Normally, when a biological organism having a complex vascular structure densely distributed within an imaging space is imaged by using the image reconfiguration algorithm of the photoacoustic apparatus, artifacts derived from a plurality of targets are superposed even in a voxel corresponding to a position where a target (blood vessel) does not exist. In addition, as the relative position between the reception position of the photoacoustic wave and the voxel changes, the strength and the degree of contribution that an artifact derived from each structural body has in the voxel changes. In other words, in a certain piece of photoacoustic image data, an image value corresponding to a position where a target does not exist is expressed by the sum of a plurality of random variables of artifacts derived from a plurality of structural bodies.

Furthermore, when the distribution of an image value group of a plurality of pieces of photoacoustic image data corresponding to a position where a target does not exist is checked, the result obtained indicates that the distribution becomes more similar to the normal distribution with increasing number of pieces of photoacoustic image data. This is an indication of the central limit theorem in an artifact of the photoacoustic image data. In this case, it can be expressed that an image value corresponding to a position where a target does not exist behaves randomly.

On the other hand, since a variation in an image value corresponding to a position where a target does not exist has a non-random behavior having a certain tendency, the distribution of an image value group tends to deviate from the normal distribution. In this case, it can be evaluated that the distribution of an image value group at a position where a target exists has non-Gaussian characteristics.

Thus, in this embodiment, it can be determined whether or not a target exists at a certain position based on the characteristics of an image value group of a plurality of pieces of photoacoustic image data at the certain position. Specifically, in this embodiment, it can be determined whether the possibility of the existence of a target at a certain position is high or low. The computer 150 determines whether the distribution of the image value group of the plurality of pieces of photoacoustic image data at the certain position is normal (Gaussian, random, or non-Gaussian), so as to distinguish between a real image and an artifact.

An example of an indicator for evaluating a normal distribution will be described. For example, an indicator called entropy may be used as the indicator for evaluating a normal distribution. Entropy, which means disorder, is known to increase in value as the randomness of a random variable increases. It is said that entropy reaches its maximum when a certain random variable is a normal distribution function. Therefore, in this embodiment, an entropy value may preferably be used as characteristic information (statistic value) based on which a real image and an artifact can be distinguished from each other. For example, entropy is expressed with expression (4) indicated below, where Pᵢ denotes a probability that an image value is equal to i. Specifically, Pᵢ is a value obtained by dividing the number of pieces of image data existing in a class where the image value is equal to i by the total number of pieces of image data. The entropy expressed with expression (4) is an indicator that is also called an average amount of information.

$$H = - {\sum }_{i} {P}_{i} {log}_{2} {P}_{i} \qquad ( 4 )$$

The characteristic information may be information indicating the characteristics of the shape of a histogram of an image value group. The information indicating the characteristics of the shape of a histogram may be information containing at least one of kurtosis and skewness of the histogram. Kurtosis is an indicator used as an assessment measure for non-Gaussian characteristics of a probability distribution and is useful for determining whether or not a target exists in this embodiment. For example, the computer 150 identifies pixels or voxels of a plurality of pieces of image data corresponding to a certain position in a two-dimensional or three-dimensional space. The computer 150 can then convert the image value group of the pixels or voxels into a histogram. In this case, the number of pieces of sample data to be converted into a histogram is equal to the number of pieces of image data.

Furthermore, the computer 150 may compare a value indicating the characteristic information with a threshold value, and may determine whether or not a target exists at a certain position depending on whether the value indicating the characteristic information is higher or lower than the threshold value.

Typically, with regard to a median value, an average value, a standard deviation value, or a variance value of an image value group, the possibility of the existence of a target at a certain position tends to increase with increasing value at the position. With regard to entropy, the possibility of the existence of a target at a certain position tends to increase with decreasing value at the position. With regard to kurtosis of a histogram, the possibility of the existence of a target at a certain position tends to increase with increasing value at the position. With regard to skewness of a histogram, the possibility of the existence of a target at a certain position tends to increase with increasing absolute value at the position.

For example, the computer 150 identifies pixels or voxels of a plurality of pieces of image data corresponding to a certain position in a two-dimensional or three-dimensional space. The computer 150 then converts the image value group of the pixels or voxels into a histogram. In this case, the number of pieces of sample data to be converted into a histogram is equal to the number of pieces of image data. The certain position may be a position designated by the user via the input unit 170, or may be a preset position. Moreover, the certain position may be a plurality of set positions.

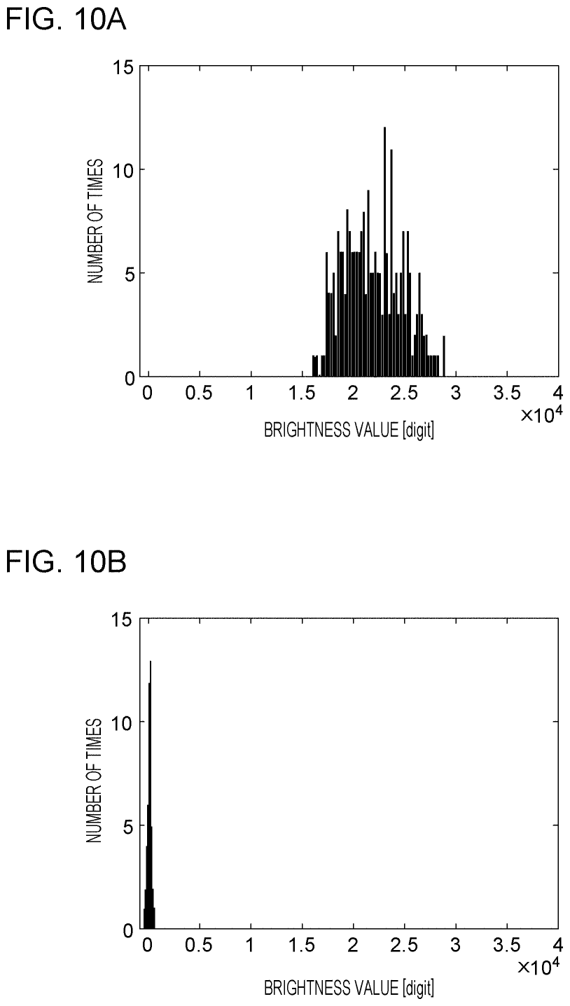

The following description relates to a case where a plurality of pieces of image data are generated in accordance with a simulation performed on the subject model 1000 shown in FIG. 4. FIGS. 10A and 10B each illustrate an example of a histogram of an image value group of a plurality of pieces of image data at a certain position, obtained as a result of a simulation. FIG. 10A illustrates a case where the image value group of the plurality of pieces of image data at the position of the blood vessel 1011 in FIG. 4 is converted into a histogram. FIG. 10B illustrates a case where the image value group of the plurality of pieces of image data at a position distant from the blood vessel 1011 by 2.5 mm is converted into a histogram. It is comprehensible from each of FIGS. 10A and 10B that there is a difference in the histogram of the image value group between a position where a target exists and a position where a target does not exist.

For example, according to the histogram shown in FIG. 10A, the kurtosis corresponding to a voxel existing in a target region is 1E-65. On the other hand, according to the histogram shown in FIG. 10B, the kurtosis corresponding to a voxel existing in a region outside the target region is 1E-70. Accordingly, it is comprehensible that the kurtosis in the target region is higher than the kurtosis in the region outside the target.

Furthermore, when a threshold value is to be set, a plurality of threshold values may be set instead of a single threshold value as a reference, such as providing a threshold value for differentiating the target region and a threshold value for differentiating the region outside the target region. Moreover, it may freely be set whether to determine that a region is a target region if the value indicating the characteristic information exceeds the threshold value or to determine that a region is a target region if the value is higher than or equal to the threshold value.

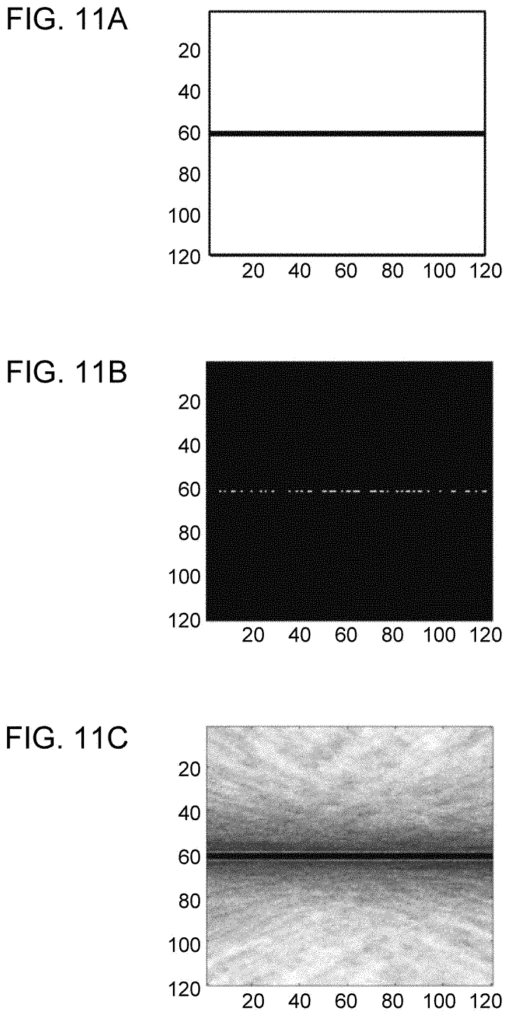

FIGS. 11A to 11C each illustrate a characteristic information image obtained by converting characteristic information corresponding to each of a plurality of positions into an image. A characteristic information image is a spatial distribution image in which a value indicated by the characteristic information corresponding to each of the plurality of positions is plotted to the corresponding position.

FIG. 11A is a cross-sectional view taken along an XY plane of the 0.2 [mm] blood vessel 1011 extending in the Y-axis direction at a depth of 20 [mm] from the surface of the subject model shown in FIG. 4.

FIG. 11B illustrates a kurtosis image obtained by calculating and plotting kurtosis for each voxel in image data corresponding to a plurality of radiated light beams and obtained by being reconfigured based on UBP described in NPL 1. In the kurtosis image shown in FIG. 11B, it is comprehensible that an image indicating high kurtosis exists discretely in the region of the blood vessel 1011. The image with high kurtosis is not included in a region outside the blood vessel 1011. It is comprehensible that the kurtosis is substantially zero (black) in the region outside the blood vessel 1011. Specifically, it is comprehensible that the possibility of the existence of a target is extremely high in a region where the kurtosis is higher than a certain threshold value.

FIG. 11C is an entropy image obtained by calculating and plotting entropy for each voxel in image data corresponding to a plurality of radiated light beams and obtained by being reconfigured based on UBP described in NPL 1. In the entropy image shown in FIG. 11C, the blood vessel 1011 is numerically converted into substantially zero (black). On the other hand, a region outside the blood vessel 1011 has a numerical value (gray) dominantly larger than zero. However, it is comprehensible that the value varies greatly, as compared with the kurtosis image, in the region outside the blood vessel 1011 in the entropy image.

Accordingly, it is comprehensible that the determination accuracy varies between a target region and a region outside the target region depending on the type of characteristic information. Based on a plurality of pieces of characteristic information of different types, the computer 150 may determine whether or not a target exists at a certain position and may acquire the determination information.

For example, in a region where the kurtosis is higher than a first threshold value, the computer 150 may determine that the region has a target existing therein regardless of the value of the entropy. Moreover, in a region where the kurtosis is lower than the first threshold value and the entropy is lower than a second threshold value, the computer 150 may determine that the region has a target existing therein. In a region where the kurtosis is lower than the first threshold value and the entropy is higher than the second threshold value, the computer 150 may determine that the region has a target existing therein.

If a continuous region where the entropy is lower than the second threshold value includes a region where the kurtosis is higher than the first threshold value, the computer 150 may determine that the continuous region is a region where a target exists.

By determining whether or not a target exists by combining pieces of characteristic information of different types in this manner, the determination accuracy can be improved.

When acquiring characteristic information, the computer 150 may use all pieces of image data or may use a plurality of selectively extracted pieces of image data.

The algorithm for differentiating whether or not there is a target is not limited to a specific type so long as it is possible to determine from a plurality of pieces of image data that noteworthy pixels or voxels are located in a target region or in a region outside the target region. The computer 150 may alternatively use an artificial intelligence algorithm to determine whether or not there is a target.

In the characteristic information, information used for the determination of a target region and a region outside the target region may be designated by the user or may be set to predetermined information by the computer 150.

(S940: Step for Generating image Using Determination Information)

The computer 150 acquires image data based on the signal data acquired in step S400. For example, the computer 150 may combine the plurality of pieces of image data acquired in step S920 so as to generate new image data (combined image data). The combining process may be, for example, an adding process, an adding/averaging process, a weighting/adding process, or a weighting/adding/averaging process.

Furthermore, the computer 150 may generate the new image data by performing reconfiguration using the plurality of pieces of signal data, obtained in step S400, corresponding to the plurality of radiated light beams.

Subsequently, the computer 150 may use the determination information acquired in step S930 to generate an image used for identifying whether or not a position has a target existing therein, and may cause the display unit 160 to display the image.

The computer 150 may perform image processing on the image data based on the determination information to generate the image used for identifying whether or not a position has a target existing therein, and may cause the display unit 160 to display the image.

For example, based on the determination information, the computer 150 may perform an amplification process by multiplying a brightness value corresponding to an image value of a pixel or voxel corresponding to a position where a target exists by a coefficient of 1 or larger. Furthermore, based on the determination information, the computer 150 may perform an attenuation process by multiplying a brightness value corresponding to an image value of a pixel or voxel corresponding to a position where a target exists by a coefficient smaller than 1. The attenuation process may involve multiplying a brightness value corresponding to an image value of the relevant pixel or voxel by 0 and substantially not displaying a region outside the target region.

Furthermore, the computer 150 may display a position where a target exists and a position where a target does not exist in different colors in a color-coded manner. In this case, the image at the position where the target exists may be displayed in a color with relatively high visibility, and the image at the position where the target does not exist may be displayed in a color with relatively low visibility.

Furthermore, the computer 150 may combine the brightness-value amplification/attenuation process and the color coding process.

The computer 150 may divide the image into three regions, namely, a target region, a region outside the target region, and a region near the boundary between the target region and the region outside the target region, and may display an image such that the respective regions are identifiable. The region near the boundary is a part of the target region or the region outside the target region.

For example, the computer 150 may perform an attenuation process involving multiplying the brightness value corresponding to the image value of the region near the boundary between the target region and the region outside the target by a coefficient smaller than 1. Then, the computer 150 may perform an amplification process involving multiplying the brightness value corresponding to the image value of the target region (excluding the region near the boundary) by a coefficient of 1 or larger, and may multiply the brightness value corresponding to the image value of the region outside the target region (excluding the region near the boundary) by 0 so as not to display the image. By performing such processes, the image of the target region and the image of the region outside the target region can be smoothly connected. Alternatively, the three regions may be displayed in different colors in a color-coded manner.

Although image display based on a single piece of image data is described in the above example, the image processing may be performed on a plurality of pieces of image data. For example, the plurality of pieces of image data may be classified into several groups each containing at least one piece of image data, and image processing may be performed on partially-combined image data generated as a result of performing a combining process individually on each group.

Furthermore, the computer 150 may display an image to which the above-described image processing is applied and an image to which the above-described image processing is not applied in a side-by-side fashion, a superposed fashion, or an alternating fashion. For example, when the computer 150 causes the display unit 160 to display the image to which the above-described image processing is not applied in step S600, the computer 150 may switch the display mode to the side-by-side display mode or the superposed display mode by receiving a command from the user for switching the display mode. Furthermore, when the computer 150 causes the display unit 160 to display the image to which the above-described image processing is not applied in step S600, the computer 150 may switch the display mode to the image to which the above-described image processing is applied by receiving a command from the user for switching the display mode via the input unit 170.

Furthermore, the computer 150 may cause the display unit 160 to display, together with an image based on image data, an image indicating characteristic information corresponding to a position designated by the user via the input unit 170. In this case, the position where the image indicating the characteristic information is displayed may be designated on the basis of a command given to the image based on the image data displayed on the display unit 160.

Moreover, the computer 150 may cause a characteristic information image converted from characteristic information corresponding to each of a plurality of positions to be displayed, as shown in FIG. 11B or 11C. Alternatively, the computer 150 may cause an image obtained by combining a plurality of different types of characteristic information images to be displayed, or may cause the plurality of types of characteristic information images to be displayed in a side-by-side fashion, superposed fashion, or an alternating fashion.

The computer 150 may cause information (e.g., a graph) indicating variations of an image value to be displayed, as shown in FIG. 3.

According to this embodiment, an image in which a target region and a region outside the target region can be easily differentiated from each other can be provided. The user checks the image displayed as in this embodiment so as to easily differentiate whether or not a target (observation target) exists at a certain position within the image.

### Other Embodiments

The present invention is also realized by executing the following process. Specifically, the process involves supplying software (program) for realizing the functions of the above-described embodiment to a system or an apparatus via a network or any of various types of storage media, and causing a computer (such as a CPU or an MPU) in the system or the apparatus to read and execute the program.

The present invention is not limited to the above-described embodiments, and various modifications and alterations are possible so long as they do not depart from the spirit and scope of the invention. Accordingly, the following claims are attached for publicizing the scope of the invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

The image generating apparatus according to the present invention can facilitate the differentiation of whether the possibility of the existence of a target (observation target) at a certain position within an image is high or low.

1. An image generating apparatus that generates image data based on a reception signal obtained by receiving a photoacoustic wave generated from a subject as a result of radiating light onto the subject, the image generating apparatus comprising:
an image data generator configured to generate a plurality of pieces of image data corresponding to a plurality of radiated light beams based on a plurality of the reception signals obtained by radiating light a plurality of times onto the subject;
a characteristic-information acquiring unit configured to acquire characteristic information indicating characteristics of an image value group of the plurality of pieces of image data at a certain position; and
an information acquiring unit configured to acquire information indicating a possibility that a target exists at the certain position based on the characteristic information.

2. The image generating apparatus according to claim 1, further comprising:
a display controller,
wherein the image data generator generates combined image data based on the plurality of reception signals,
wherein, if the target exists at the certain position based on the information, the display controller causes a display unit to display an image based on the combined image data at the certain position, the image being displayed with a brightness corresponding to an image value of the combined image data at the certain position, and
wherein, if the target does not exist at the certain position based on the information, the display controller causes the display unit to display the image based on the combined image data at the certain position, the image being displayed with a brightness lower than the brightness.

3. The image generating apparatus according to claim 1, further comprising:
a display controller configured to cause a display unit to display an image based on the image data with which the possibility that the target exists at the certain position based on the information is differentiable.

4. An image generating apparatus that generates image data based on a plurality of reception signals corresponding to a plurality of radiated light beams and obtained by receiving a photoacoustic wave generated from a subject as a result of radiating light a plurality of times onto the subject, the image generating apparatus comprising:
an image data generator configured to generate a plurality of pieces of image data corresponding to the plurality of radiated light beams based on the plurality of reception signals;
a characteristic-information acquiring unit configured to acquire characteristic information indicating characteristics of an image value group of the plurality of pieces of image data at a certain position; and
a display controller,
wherein the image data generator generates combined image data based on the plurality of reception signals, and
wherein the display controller causes a display unit to display an image including a first image based on the combined image data and a second image based on the characteristic information.

5. The image generating apparatus according to claim 2,
wherein the image data generator generates the combined image data by combining the plurality of pieces of image data.

6. The image generating apparatus according to claim 1, further comprising:
a signal processor configured to perform signal processing including a time differentiation process on each of the plurality of reception signals,
wherein the image data generator generates the plurality of pieces of image data based on the plurality of reception signals having undergone the signal processing.

7. The image generating apparatus according to claim 6,
wherein the signal processor performs the signal processing on each of the plurality of reception signals, the signal processing including the time differentiation process and an inversion process for inverting positive and negative signs of signal levels.

8. The image generating apparatus according to claim 1,
wherein the characteristic-information acquiring unit acquires a plurality of pieces of the characteristic information of different types, and
wherein the information acquiring unit acquires the information indicating the possibility that the target exists at the certain position based on the plurality of pieces of characteristic information.

9. The image generating apparatus according to claim 8,
wherein the plurality of pieces of characteristic information include kurtosis of a histogram of the image value group and entropy of the image value group,
wherein the information acquiring unit acquires the information by determining that the target exists at the certain position when the kurtosis is higher than a first threshold value,
wherein the information acquiring unit acquires the information by determining that the target exists at the certain position when the kurtosis is lower than the first threshold value and the entropy is lower than a second threshold value, and
wherein the information acquiring unit acquires the information by determining that the target does not exist at the certain position when the kurtosis is lower than the first threshold value and the entropy is higher than the second threshold value.

10. The image generating apparatus according to claim 1,
wherein the characteristic information includes information indicating a shape of a histogram of the image value group.

11. The image generating apparatus according to claim 1,
wherein the characteristic information includes a statistic value of the image value group.

12. The image generating apparatus according to claim 1,
wherein the characteristic information includes at least one of an average value, a standard deviation value, a variance value, entropy, negentropy, kurtosis, and skewness of the image value group.

13. An image generating method for generating image data based on a plurality of reception signals corresponding to a plurality of radiated light beams and obtained by receiving a photoacoustic wave generated from a subject as a result of radiating light a plurality of times onto the subject, the image generating method comprising:
generating a plurality of pieces of image data based on the plurality of reception signals;
acquiring characteristic information indicating characteristics of an image value group of the plurality of pieces of image data at a certain position; and
acquiring information indicating a possibility that a target exists at the certain position based on the characteristic information.

14. The image generating method according to claim 13, further comprising:
generating combined image data based on the plurality of reception signals;
causing a display unit to display an image based on the combined image data at the certain position if the target exists at the certain position based on the information, the image being displayed with a brightness corresponding to an image value of the combined image data at the certain position; and
causing the display unit to display the image based on the combined image data at the certain position if the target does not exist at the certain position based on the information, the image being displayed with a brightness lower than the brightness.

15. The image generating method according to claim 13, further comprising:
causing a display unit to display an image based on the image data with which the possibility that the target exists at the certain position based on the information is differentiable.

16. An image generating method for generating image data based on a plurality of reception signals corresponding to a plurality of radiated light beams and obtained by receiving a photoacoustic wave generated from a subject as a result of radiating light a plurality of times onto the subject, the image generating method comprising:
generating a plurality of pieces of image data corresponding to the plurality of radiated light beams based on the plurality of reception signals;
acquiring characteristic information indicating characteristics of an image value group of the plurality of pieces of image data at a certain position;
generating combined image data based on the plurality of reception signals; and
causing a display unit to display an image including a first image based on the combined image data and a second image based on the characteristic information.

17. The image generating method according to claim 13, further comprising:
acquiring a plurality of pieces of the characteristic information of different types; and
acquiring the information indicating the possibility that the target exists at the certain position based on the plurality of pieces of characteristic information.

18. The image generating method according to claim 17,
wherein the plurality of pieces of characteristic information include kurtosis of a histogram of the image value group and entropy of the image value group,
wherein the information is acquired by determining that the target exists at the certain position when the kurtosis is higher than a first threshold value,
wherein the information is acquired by determining that the target exists at the certain position when the kurtosis is lower than the first threshold value and the entropy is lower than a second threshold value, and
wherein the information is acquired by determining that the target does not exist at the certain position when the kurtosis is lower than the first threshold value and the entropy is higher than the second threshold value.

19. A non-transitory computer-readable medium storing a program for causing a computer to execute the image generating method according to claim 13.
